(19) United States
(12) Patent Application Publication
(10) Pub. No.: US 2011/0268668 A1
(43) Pub. Date: Nov. 3, 2011

(54) PROCESS FOR NEBULIZING AQUEOUS COMPOSITIONS CONTAINING HIGHLY CONCENTRATED INSULIN

(76) Inventors: Herbert Lamche, Alland (AT); Christopher John Montague Meade, Maselheim (DE); Bernd Zierenberg, Bingen am Rhein (DE); Ralph Christian Reimholz, Wiesbaden (DE)

(21) Appl. No.: 13/183,057

(22) Filed: Jul. 14, 2011

Related U.S. Application Data
(63) Continuation of application No. 11/456,564, filed on Jul. 10, 2006, now abandoned.
Application No. 11/456,564 is a continuation of application No. 10/288,770, filed on Nov. 6, 2002, now abandoned.
Application No. 10/288,770 is a continuation of application No. 09/497,696, filed on Feb. 3, 2000, now abandoned.
Application No. 09/497,696 is a continuation-in-part of application No. PCT/EP98/04803, filed on Jul. 31, 1998.

(30) Foreign Application Priority Data
Aug. 4, 1997 (DE) 197 33 651.5

(51) Int. Cl. A61K 9/12; A61P 3/10; A61K 38/28

(52) U.S. Cl. 424/45; 514/5.9

(57) ABSTRACT

A process for nebulizing highly concentrated solutions of insulin for administration by inhalation.

### RELATED APPLICATIONS

This application is a continuation of U.S. application Ser. No. 09/497,696, now abandoned, which was a continuation-in-part, under 35 U.S.C. §365(c), of International Application No. PCT/EP98/04803, filed Jul. 31, 1998.

### FIELD OF THE INVENTION

The invention relates to a process for producing aerosols for administration of proteins and other biologically active macromolecules by inhalation, as well as aqueous preparations for producing such aerosols. In particular, the invention relates to aqueous preparations of highly concentrated solutions of insulin for administration by inhalation for the treatment of diabetes.

### BRIEF DESCRIPTION OF THE DRAWINGS

FIG. 1 shows an in vitro system for testing a nebulizer in which a protein solution is nebulized and the spray mist is caught in a so-called “trap”;

FIGS. 2a and 2b are graphs showing the interferon omega levels measured after 20 puffs of interferon omega from the RESPIMAT® device, measured by immunological methods (FIG. 2a) and biological methods (FIG. 2b);

FIG. 3 is a graph showing measured neopterin levels after administration of interferon omega by the RESPIMAT® device and after intravenous administration of 0.32 mg of interferon, showing that administration by inhalation yielded significantly higher and longer lasting levels than intravenous administration;

FIGS. 4a and 4b, which are identical to FIGS. 6a and 6b in WO 97/12687, show the RESPIMAT® nebulizer with which the aqueous aerosol preparations according to the invention may advantageously be inhaled: FIG. 4a shows a longitudinal section through the atomizer with the spring biased and FIG. 4b shows a longitudinal section through the atomizer with the spring released; and

FIG. 5 is a graph showing the measured blood glucose level in a dog after administration with highly concentrated insulin solution by inhalation, as described herein.

### BACKGROUND AND DESCRIPTION OF THE INVENTION

It has long been known to administer drugs in the form of inhalable aerosols. Aerosols of this kind are used not only to treat respiratory disorders such as asthma but are also used when the lungs or the nasal mucous membranes are intended to act as an organ of absorption. Frequently, blood levels of the active substance are achieved which are high enough to treat diseases in other parts of the body. Inhalable aerosols may also be used as vaccines.

In practice, numerous methods are used for producing aerosols. Either suspensions or solutions of active substances are sprayed with the aid of propellant gases, or active substances in the form of micronised powders are fluidized in the air breathed in or, finally, aqueous solutions are atomized using nebulizers.

However, in the case of molecules of complex structure such as interferons, the nebulization of aqueous solutions can readily lead to a serious reduction in the activity of the active substance, presumably as the result of shear forces and heating. It is thought that the formation of less active protein aggregates, for example, plays a part in this process. In their article “Stability of Recombinant Consensus Interferon to Airjet and Ultrasonic Nebulization”, J. Pharm. Sci. 84: 1210-1215 (1995), A. Y. Ip and colleagues described examples of the formation of interferon aggregates after ultrasound or jet nebulization, with the concomitant loss of the biological activity of the interferon. Even if the biomolecule (biologically active macromolecule) is not completely destroyed, the loss of activity described here is important as it constitutes a fairly large consumption of the possibly expensive biomolecule and leads to inaccurate dosing of active substance per actuation. This reduction in activity of molecules of complex structure during the production of the aerosol is not restricted to interferons; it also occurs to a greater or lesser extent when other proteins (c.f., for example, Niven et al., Pharm. Res. 12: 53-59 (1995)) and biomolecules are made into aerosol form.

Apart from the industrial production of the aerosol which contains the biomolecule, a second step is needed to ensure that the biomolecules are absorbed into the lungs. The lung of an adult human presents a large surface area for absorption but also has a number of obstacles to the pulmonary absorption of biomolecules. After being breathed in through the nose or mouth, air together with the aerosol it carries passes into the trachea and then through smaller and smaller bronchi and bronchioles into the alveoli. The alveoli have a much larger surface area than the trachea, bronchi and bronchioles together. They are the main absorption zone, not only for oxygen but also for biologically active macromolecules. In order to pass from the air into the bloodstream, molecules have to cross the alveolar epithelium, the capillary epithelium and the lymph-containing interstitial space between these two layers of cells. This can be done through active or passive transport processes. The cells in these two layers of cells are arranged close together, so that the majority of large biological macromolecules (such as proteins) cross this barrier much more slowly than smaller molecules. The process of crossing the alveolar epithelium and capillary endothelium proceeds in competition with other biological processes which lead to the destruction of the biomolecule. The bronchoalveolar fluid contains exoproteases (cf., for example, D. A. Wall and A. T. Lanutti, “High Levels of Exopeptidase Activity are Present in Rat and Canine Bronchoalveolar Lavage Fluid”, Int. J. Pharm. 97: 171-181 (1993)). It also contains macrophages which eliminate protein particles by phagocytosis. These macrophages migrate to the base of the bronchial tree, from where they leave the lungs by means of the mucociliary clearance mechanism. They are then able to migrate into the lymphatic system. Moreover, the macrophages may be influenced in their physiology by the protein in aerosol form, e.g., interferons may activate alveolar macrophages. The migration of activated macrophages is another mechanism for propagating the systemic effect of an inhaled protein. The complexity of this process means that results of aerosol tests with one type of protein can only be transferred to another type of protein to a limited degree. Small differences between interferons may, for example, have a significant effect on their susceptibility to the degradation mechanisms in the lungs (see Bocci et al., “Pulmonary Catabolism of Interferons: Alveolar Absorption of ¹²⁵I-Labelled Human Interferon Alpha is Accompanied by Partial Loss of Biological Activity”, Antiviral Res. 4: 211-220 (1984)).

Proteins and other biological macromolecules may indeed by nebulized in theory but as a rule this nebulization is accompanied by a loss of activity. The objective of the present invention is to provide a process for producing inhalable aerosols by means of which biologically active macromolecules, particularly proteins, can be nebulized without any substantial loss of activity.

A new generation of propellant-free nebulizers is described in U.S. Pat. No. 5,497,944; reference is hereby made to the contents of this patent. The particular advantage of the nebulizers described therein is that there is no need to use propellant gases, particularly fluorochlorohydrocarbons.

A more developed embodiment of the nebulizers described therein is disclosed in PCT/EP96/04351=WO 97/12687. Regarding the present invention, reference is made specifically to FIG. 6 described therein (the RESPIMAT® device) and to the associated parts of the description of this application. The nebulizer described therein can advantageously be used to produce the inhalable aerosols of biologically active macromolecules according to the invention. In particular, the nebulizer described therein can be used for the inhalation of insulin. Thanks to its convenient size, this device can be carried around by the patient at all times. With the nebulizer described, active substance-containing solutions of specified volumes (preferably about 15 μL) are sprayed under high pressure through small nozzles so as to form inhalable aerosols with an average particle size of between 3 and 10 microns. For the inhalation of insulin, nebulizers which are able to nebulize between 10 μL and 50 μL of aerosol preparation per application into inhalable droplets are suitable.

A feature which is of particular importance in the preparation of the aerosols according to the invention is the use of the nebulizer described in the above mentioned patent or patent application for the propellant-free atomization of solutions of active substance which contain proteins or other biologically active macromolecules.

Essentially, the conveniently sized atomizer disclosed therein (nebulizer, about 10 cm in size) consists of an upper housing part, a pump housing, a nozzle, a clamping mechanism, a spring housing, a spring and a reservoir container, characterized by:
a pump housing fixed in the upper housing part and bearing at one end a nozzle member with the nozzle or nozzle arrangement,
a hollow piston with valve member,
a drive flange in which the hollow piston is fixed and which is located in the upper housing part,
a clamping mechanism located in the upper housing part,
a spring housing with the spring located therein, which is rotatably mounted by a rotary bearing on the upper housing part, and
a lower housing part which is fitted on the spring housing in the axial direction.

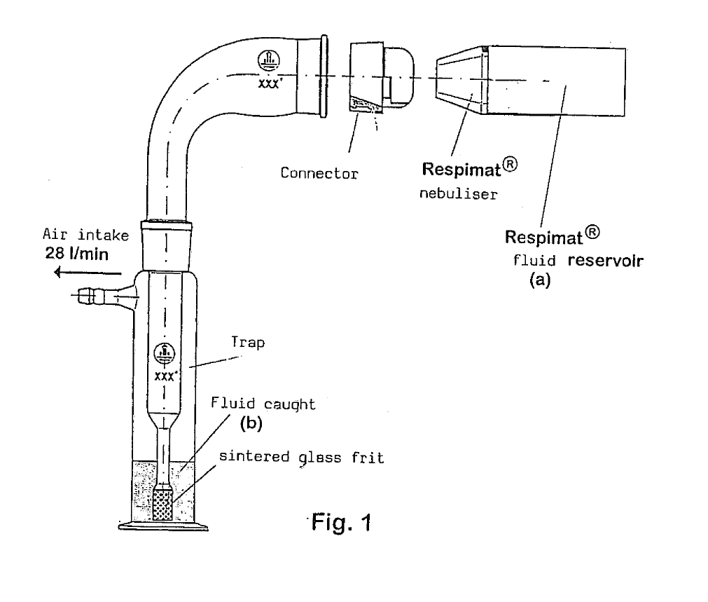

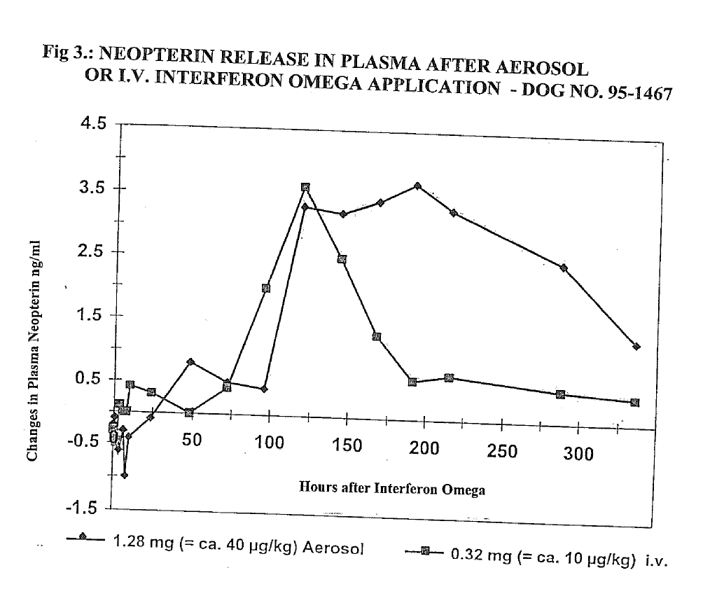

The hollow piston with valve member WO 97/12687 corresponds to one of the devices disclosed. It projects partly into the cylinder of the pump housing and is mounted so as to be axially movably within the cylinder. Reference is made particularly to FIGS. 1 to 4 thereof, especially FIG. 3, and the associated parts of the specification. The hollow piston with valve member exerts a pressure of 5-60 MPa (about 50-600 bar), preferably 10-60 MPa (about 100-600 bar) on the fluid, the appropriate solution of active substance, on its high pressure side at the time of release of the spring.

The valve member is preferably mounted on the end of the hollow piston facing the nozzle member.

The nozzle in the nozzle member is preferably microstructured, i.e., produced by microtechnology. Microstructured nozzle members are disclosed, for example, in WO 94/07607; reference is hereby made to the contents of this specification.

The nozzle member consists, for example, of two plates of glass and/or silicon firmly attached to each other, at least one plate of which has one or more microstructured channels which connect the inlet side of the nozzle to the outlet side. On the outlet side of the nozzle is provided at least one round or non-round opening smaller than or equal to 10 microns.

The directions of flow of the nozzles in the nozzle member may run parallel to one another or be inclined relative to one another. In the case of a nozzle member having at least two nozzle openings on the outlet side, the directions of flow may be inclined at an angle of 20° to 160° to one another, preferably at an angle of from 60° to 150°. The directions of flow meet in the vicinity of the nozzle openings.

The clamping mechanism contains a spring, preferably a cylindrical helical compression spring, as a store of mechanical energy. The spring acts on the drive flange as a jumping member, the movement of which is determined by the position of a locking member. The path of the drive flange is precisely bounded by an upper and a lower stop. The spring is preferably put under tension, via a force-transmitting gear, e.g., a helical thrust cam, by an external torque which is produced as the upper part of the housing is rotated counter to the spring housing in the lower housing part. In this case, the upper housing part and the drive flange contains a single- or multi-speed wedge gear.

The locking member with engaging locking surfaces is arranged in an annular configuration around the drive flange. It consists, for example, of a plastics or metal ring which has intrinsic radial elastic deformability. The ring is arranged in a plane at right angles to the atomizer axis. After the tensioning of the spring the locking surfaces of the locking member slide into the path of the drive flange and prevent the spring from being released. The locking member is actuated by means of a button. The actuating button is connected or coupled to the locking member. In order to actuate the locking mechanism the actuating button is pushed parallel to the plane of the ring, preferably into the atomizer; the deformable ring is thus deformed in the plane of the ring. Details of the locking values are described in WO 97/20590.

The lower housing part is pushed axially over the spring housing and covers the bearing, the drive of the spindle and the reservoir container for the fluid.

When the atomizer is operated, the upper housing part is rotated counter to the lower housing part, whilst the lower housing part takes the spring housing with it. The spring is compressed and biased by means of the helical thrust cam and the locking mechanism engages automatically. The angle of rotation is preferably a whole-number fraction of 360°, e.g. 180°. At the same time as the spring is biased, the drive member in the upper housing part is moved a given distance, the hollow piston is pulled back within the cylinder in the pump housing, as a result of which some of the fluid from the reservoir container is sucked into the high pressure chamber in front of the nozzle.

If the desired, a plurality of exchangeable reservoir containers containing the fluid to be atomized may be inserted into the atomizer and used. The reservoir container contains the aqueous aerosol preparation according to the invention.

The atomizing process is started by gently pressing the actuating button. The locking mechanism then opens up the way for the drive member. The biased spring pushes the piston into the cylinder of the pump housing. The fluid leaves the atomizer nozzle in spray form.

Other details of construction are disclosed in PCT applications WO 97/12683 and WO 97/20590; reference is hereby made to the contents of these publications.

The components of the atomizer (nebulizer) are made of a material suitable for the purpose. The housing of the atomizer and, as far as its operation permits, other parts are preferably made of plastics, e.g., by injection moulding. For medical purposes, physiologically acceptable materials are used.

The atomizer described in WO 97/12687 is used, for example, for propellant-free production of medicinal aerosols. An inhalable aerosol with an average droplet size of about 5 μm can be produced therewith.

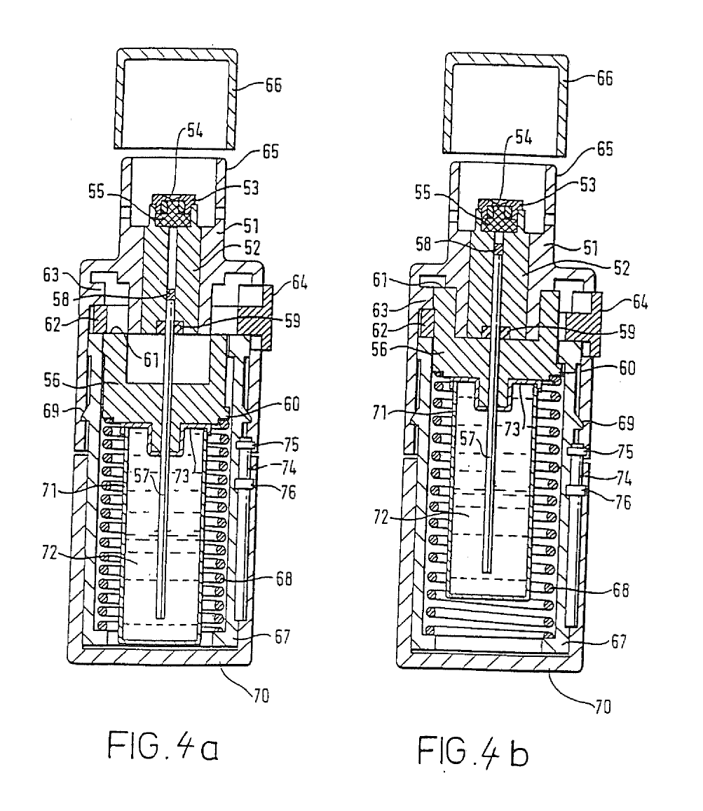

FIGS. 4a and 4b, which are identical to FIGS. 6a and 6b in WO 97/12687, show the nebulizer (RESPIMAT®) with which the aqueous aerosol preparations according to the invention may advantageously be inhaled.

FIG. 4a shows a longitudinal section through the atomizer with the spring biased, FIG. 4b shows a longitudinal section through the atomizer with the spring released.

The upper housing part (51) contains the pump housing (52) on the end of which is mounted the holder (53) for the atomizer nozzle. In the holder are located the nozzle member (54) and a filter (55). The hollow piston (57) secured in the drive flange (56) of the clamping mechanism projects partly into the cylinder of the pump housing. At its end, the hollow piston carries the valve member (58). The hollow piston is sealed off by means of the seal (59). Inside the upper housing part is the stop (60) on which the drive flange rests when the spring is released. On the drive flange is the stop (61) on which the flange rests when the spring is biased. After the spring has been biased, the locking member (62) moves between the stop (61) and a support (63) in the upper housing part. The actuating button (64) is connected to the locking member. The upper housing part ends in the mouth piece (65) and is closed off by means of the removable protective cover (66).

The spring housing (67) with compression spring (68) is rotatably mounted by means of the snap-fit lugs (69) and rotary bearings on the upper housing part. The lower housing part (70) is pushed over the spring housing. Inside the spring housing is located the exchangeable reservoir container (71) for the fluid (72) which is to be atomized. The reservoir container is closed off by means of the stopper (73) through which the hollow piston projects into the storage container and dips its ends into the fluid (supply of active substance solution).

The spindle (74) for the mechanical counter is mounted in the outer surface of the spring housing. On the end of the spindle facing the upper housing part is the drive pinion (75). The slider (76) rests on the spindle.

The nebulizer described above is suitable for nebulizing the aerosol preparations according to the invention to produce an aerosol suitable for inhalation.

The effectiveness of a nebulizer can be tested using an in vitro system in which a protein solution is nebulized and the spray mist is caught in a so-called “trap” (see FIG. 1). The activity of the protein in the aerosol reservoir (a) is compared with its activity in the trapped liquid (b), e.g., by means of an immunoassay or using an assay for the biological effectiveness of the protein. This experiment makes it possible to evaluate the degree of destruction of the protein by the nebulizing process. A second parameter of the aerosol quality is the so-called inhalable proportion, which is defined here as the proportion of the mist droplets with a measured median aerodynamic diameter (MMAD) of less than 5.8 μm. The inhalable proportion can be measured using an “Andersen Impactor”. For good protein absorption it is important not only to achieve nebulization without any substantial loss of activity but also to generate an aerosol with a good inhalable proportion (about 60%). Aerosols with an MMAD of less than 5.8 μm are significantly better suited to reaching the alveoli, where their chances of being absorbed are significantly greater. The effectiveness of a nebulization device can also be tested in an in vivo system; in this case factors such as susceptibility to lung proteases come into play. As an example of an in vivo test system, a protein-containing mist can be administered to a dog through a tracheal tube. Blood samples arc taken at suitable time intervals and the protein level in the plasma is then measured by immunological or biological methods.

Suitable nebulizers are described in U.S. Pat. No. 5,497,944 mentioned above and in WO 97/12687, particularly as described in FIGS. 6a and 6b (here 4a and 4b). A preferred nozzle arrangement for nebulizing the aqueous aerosol preparations of biologically active macromolecules according to the invention is shown in FIG. 8 of the U.S. Patent.

Surprisingly, it has been found that the propellant-free nebulizer described above which sprays a predetermined quantity, e.g., 15 μL, of an aerosol preparation under high pressure of between 100 bar and 500 bar through at least 1 nozzle with a hydraulic diameter of 1-12 μm so as to produce inhalable droplets with an average particle size of less than 10 μm, is suitable for nebulizing liquid aerosol preparations of proteins and other macromolecules, since it is able to nebulize a broad range of proteins without any appreciable loss of activity. A nozzle arrangement as shown in FIG. 8 of the above-mentioned U.S. Patent is preferred. What is particularly surprising is the ability of nebulizers of this type to nebulize interferons which can otherwise only be nebulized with considerable loss of activity. The particularly high activity of Interferon Omega after nebulization with this device is also surprising, not only in in vitro tests but also in in vivo tests.

Another advantage of the process claimed is its surprising ability to nebulize even highly concentrated solutions of biologically active macromolecules without any substantial loss of activity. The use of highly concentrated solutions makes it possible to use a device which is small enough to be carried comfortably at all times in a jacket pocket or handbag. The nebulizer disclosed in FIG. 4 satisfies these requirements and can be used to nebulize highly concentrated solutions of biologically active molecules.

For example, devices of this kind are particularly suitable for enabling diabetics to treat themselves with insulin by inhalation. Preferably, highly concentrated aqueous solutions with a concentration of 20 to 90 mg/mL of insulin are used; solutions containing 33 to 60 mg/mL of insulin are preferred and solutions containing 33 to 40 mg/mL of insulin are particularly preferred. Depending on the size of the reservoir available in the nebulizer, solutions containing insulin in a concentration of more than 25 mg/mL, preferably more than 30 mg/mL, are suitable for inhaling a therapeutically effective quantity of insulin with a hand-held device such as the device described above. The administration of insulin by inhalation allows the active substance to start acting quickly so that the patient can treat themselves with the amount they require shortly before meal times, for example. The small size of the RESPIMAT® device, for example, makes it possible for the patient to carry the device at all times.

The RESPIMAT® device (FIG. 6 in WO 97/12687) has a dosing chamber of constant volume which enables the patient to determine and inhale the dosage of insulin which they require by the number of puffs. Apart from the number of puffs, the metering of the insulin is determined by the concentration of the insulin solution in the reservoir container (72). It may be, for example, between 25 and 90 mg/mL, with more highly concentrated solutions of about 30 mg/mL upwards being preferred.

A process for preparing highly concentrated stable insulin solutions is described, for example, in PCT applications WO 83/00288 (PCT/DK82/00068) and WO 83/03054 (PCT/DK83/00024), to which reference is hereby made.

Aerosol preparations according to the invention which contain insulin administered by the device described above should not exceed a dynamic viscosity of more than 1600×10⁻⁶ Pa·s to ensure that the inhalable proportion of the spray produced does not fall below an acceptable level. Insulin solutions with a limiting viscosity number of up to 1200×10⁻⁶ Pa·s and most preferably up to 1100×10⁻⁶ Pa·s (Pascal seconds) are preferred. If necessary, instead of using water as solvent it is possible to use solvent mixtures in order to reduce the viscosity of the medicament solution. This can be done for example by adding ethanol. The amount of ethanol in the aqueous formulation may be up to 50%, for example; an amount of 30% is preferred.

The aerosol preparation preferably has a viscosity of up to 1600×10⁻⁶ Pa·s, a range from 900 to 1100×10⁻⁶ Pa·s being particularly preferred.

Also preferred are aerosol preparations the aqueous solutions of which have a viscosity of between 900 and 1600×10⁻⁶ Pa·s, of which aqueous solutions with a viscosity in the range from 950 to 1300×10⁻⁶ Pa·s are particularly preferred.

A further objective of the present invention is to propose a suitable aerosol preparation which is appropriate for use in the processes claimed.

The invention also relates to aerosol preparations in the form of aqueous solutions which contain as active substance biologically active macromolecules, particularly a protein or peptide, in an amount of between 3 mg/mL and 100 mg/mL, preferably between 25 mg/mL and 100 mg/mL.

It has been found, surprisingly, that even higher viscosity solutions of macromolecules can be sprayed into inhalable droplets of suitable size using the process claimed according to the invention. This makes it possible to administer larger amounts of active substance per application and thus increases the therapeutic effectiveness of macromolecules in inhalation therapy.

According to the process of the invention, aqueous aerosol preparations containing macromolecules (e.g., albumin) can be used up to a viscosity of 1600×10⁻⁶ Pa·s (measured at 25° C.). At a viscosity of 1500×10⁻⁶ Pa·s an inhalable proportion of 32% was still obtained.

Higher viscosity solutions of macromolecules with a viscosity of up to 1100×10⁻⁶ Pa·s are preferred. With such solutions, an inhalable proportion of droplets containing an active substance of about 60% is obtained. The limiting viscosity numbers given were detected using an Ostwald viscosimeter using the method known from the literature. For comparison, the viscosity of water is 894×10⁻⁶ Pa·s (measured at 25° C.).

In order to illustrate advantages of the process according to the invention, the following is a description of in vitro and in vivo tests with an interferon omega solution.

### In vitro Tests With RESPIMAT® And Interferon Omega

The reservoir of a RESPIMAT® device (a) was filled with a 5 mg/mL interferon omega solution (formulated in 50 mM trisodium citrate, 150 mM NaCl, pH 5.5). The device was activated and a volume of about 12.9 μL, (one puff) was nebulized in an air current of 28 l/min. The nebulized solution was caught in a trap (FIG. 1). Interferon omega was measured in the reservoir solution and in the solution caught in the trap by immunological methods, using an ELISA, and biologically, by inhibiting the destruction of encephalo-myocarditis virus infected A549 cells. Immunological measurement of interferon is relatively simple. Published tests with nebulized proteins are restricted in many cases to immunological measurements. However, additional biological measurements are very important as they are a particularly sensitive and therapeutically relevant method of quantifying protein destruction. They do not always give the same results as physico-chemical or immunological methods because a molecule can lose its biological properties without any change in its bonding to antibodies.

In three experiments, 84%, 77%, and 98%, of the immunologically identifiable interferon, based on the starting solution, were found in the trap solution (b). Biological measurements with the same solutions gave results of 54%, 47%, and 81% recovery of the biologically identifiable interferon in the trapped solution. This very high percentage shows that nebulization with the RESPIMAT® device destroys only a relatively small amount of the activity of the interferon. The spray mist from a RESPIMAT® device as described above was also passed into an Andersen impactor by means of an air current (28 l/min). The proportion of particles less than 5.8 μm in size (“inhalable proportion”) was measured. The inhalable proportion corresponded to 70% (immunological measurements). Proteins such as interferons are often formulated with human serum albumin in order to provide further protection for the sensitive interferons. A formulation as above but with additional human serum albumin (0.5%) was also tested. In three tests, 83%, 83%, and 79%, again based on the starting solution, of the immunologically identifiable interferon were found in the trap solution (b). Biological measurements with the same solutions yielded 60%, 54%, and 66% of the biologically active interferon in the trapped solution. The inhalable proportion (immunological measurements) was 67%. In another set of tests, a concentrated interferon omega solution was poured into the reservoir of the RESPIMAT® device in a concentration of 53 mg/mL and then nebulized. In four tests, 100%, 60%, 68%, and 72%, based on the starting solution, of the immunologically identifiable interferon were found in the trapped solution (b). Biological measurements with the same solutions yielded 95%, 98%, 61%, and 83% recovery of the biologically identifiable interferon in the trapped solution. This high recovery rate shows that the

RESPIMAT® device can also be used to nebulize concentrated protein solutions without excessive losses of interferon activity.

### In Vivo Tests With RESPIMAT® And Interferon Omega

Interferon omega was administered by inhalation and intravenous route in separate experiments on the same dog. The blood levels of interferon were measured immunologically and biologically at different times. In addition, the neopterin level in the blood was measured. Neopterin is a marker for immune activation; it is released by macrophages after interferon stimulation [see Fuchs et al., “Neopterin, Biochemistry and Clinical Use as a Marker For Cellular Immune Reactions”, Int. Arch. Allergy Appl. Immunol. 101: 1-6 (1993)]. Measurement of the neopterin level serves to quantify interferon activity.

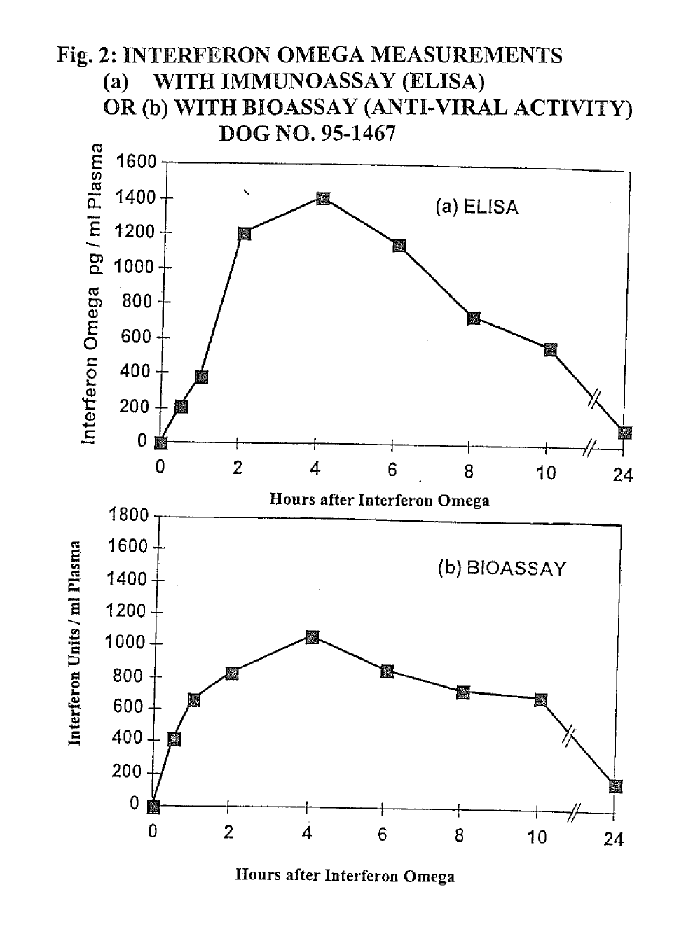

The administration of interferon to the dog was carried out under pentobarbital anesthetic after previous basic sedation. The animal was intubated and subjected to artificial ventilation (volume-controlled respiration: volume per minute 4 L/min, rate 10 breaths/min). A total of 20 puffs were delivered by the RESPIMAT® device. Each puff was given at the start of an inward breath. After the breathing in phase there were five seconds gap before breathing out. Before the next administration of interferon omega the animal was allowed to breathe for two breathing cycles without intervention. Blood for serum and heparin plasma was taken before the administration of interferon and at various times up to 14 days after the administration of interferon. Interferon omega was measured in heparin plasma by immunological methods using an ELISA and by biological methods by the inhibition of the destruction of encephalo-myocarditis virus infected A549 cells. Serum neopterin was determined by immunology. FIG. 2 shows the interferon omega levels measured after 20 puffs of interferon omega from the RESPIMAT® device, measured by immunological methods (FIG. 2a) and biological methods (FIG. 2b). Surprisingly, after administration by inhalation, a very high serum neopterin level was measured. In the test carried out in vitro, the amount of solution delivered after one puff of the RESPIMAT® device corresponded to 12.8 mg/puff, on average. Consequently, it can be expected that about 1.28 mg of interferon will be delivered by 20 puffs of the

RESPIMAT® using a 5 mg/mL solution. Neopterin measurements after the administration of this amount yielded significantly higher and longer lasting levels than neopterin measurements after intravenous administration of 0.32 mg of interferon. FIG. 3 shows this result. The high neopterin levels are evidence that the administration of interferon by the RESPIMAT® device can lead to a good biological activity.

The advantages of the RESPIMAT® device for nebulizing biologically active macromolecules is not restricted to interferon, as can be seen from a second example.

### In Vitro Tests With RESPIMAT® And Manganese Superoxide Dismutase

The device for nebulizing the test substance and the associated trap are as shown in FIG. 1. In this experiment, the reservoir (a) of the RESPIMAT® device was filled with 3.3 mg/mL of manganese superoxide dismutase (MnSOD) in phosphate-buffered saline (PBS). The device was operated and a volume of about 13 μL (one puff) was nebulized in an air current of 28 L/min. The precise amount nebulized was determined gravimetrically (measurements in three succeeding tests: 12.8, 13.7, and 14.3 mg). The nebulized solution was caught in a trap (b). This trap contained 20 mL of PBS. In addition, 2 mL of 5% bovine serum albumin was added to stabilize proteins in the trap. MnSOD was determined in the reservoir solution and in the solution caught in the trap, immunologically using an ELISA and enzymatically by the reduction in the quantity of superoxide after a xanthine/xanthine oxidase reaction. In three tests, 78%, 89%, and 83% of the immunologically identifiable MnSOD of the nebulized solution were measured in the trapped solution (b). There was no measurable loss in enzymatic activity after nebulization. The inhalable proportion (immunological measurements) was 61%.

The following example describes the production of an aerosol preparation according to the invention containing insulin as active substance.

### Preparation of the Insulin Solution And Filling the Nebulizer

175 mg of crystallized insulin (sodium salt) from cattle (corresponding to 4462.6 I.U. according to the manufacturers' information) were dissolved in 3.5 mL of sterile purified water (SERALPUR® water). Then 8.5 μL of m-cresol (corresponding to 8.65 mg) and 7.53 mg of phenol, dissolved in 100 μL of sterile purified water were added with gentle stirring. To this solution were added 365 μL of a 5 mg/mL ZnCl₂ solution (corresponding to a proportion by weight of 0.5% zinc based on the quantity of insulin used) and the pH was adjusted to 7.4 with 0.2 N NaOH. The volume of the mixture was made up to 5 mL with sterile purified water and filtered through a sterile millipore filter (pore size 0.22 μm). 4.5 mL of the aerosol preparation were transferred into the reservoir container (72, FIG. 4) of the nebulizer (RESPIMAT®). The container was closed off with a cap and the device was loaded with the container.

The aerosol preparation thus produced has a concentration of about 35 mg/mL of insulin, the viscosity of the solution being about 1020×10⁻⁶ Pa·s.

### In vivo Test With the RESPIMAT® Device And Highly Concentrated Insulin Solution

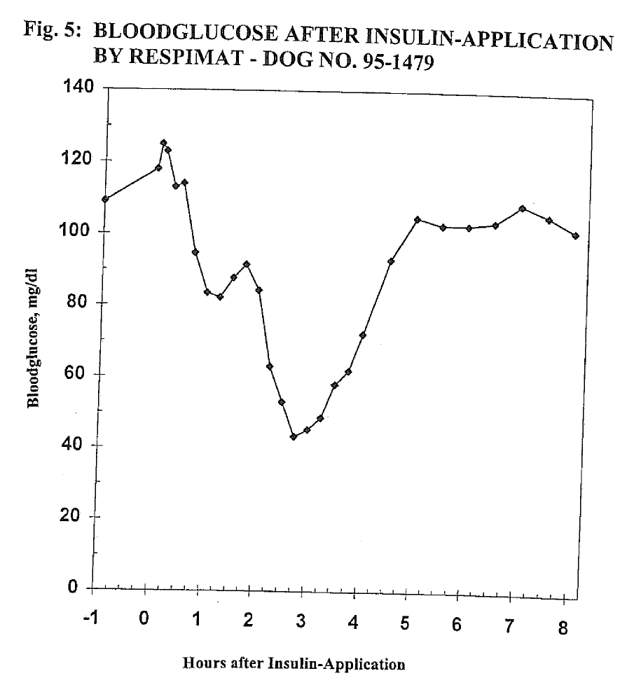

The insulin was administered to the dog anesthetized with pentobarbital after previously receiving basic sedation. The animal was intubated and ventilated as before. A total of six puffs of insulin solution were delivered from the RESPIMAT® device. Each puff was administered at the start of an inward breath. Between the breathing in phase and the breathing out phase there was a gap of 5 seconds. Before the next administration of insulin, two breath cycles were left with no intervention. Blood was taken one hour before administration, at the same time as administration and at various times thereafter over o hours. The blood glucose level was measured in the fresh blood using the method of Trasch, Koller, and Tritscher (Klein. Chem. 30; 969 [1984]) using a REFLETRON® device made by Boehringer Mannheim. Surprisingly, even with this highly concentrated insulin solution, good biological activity was obtained (lowering of blood glucose level after administration of insulin by inhalation). FIG. 5 illustrates this result.

The aqueous aerosol preparations according to the invention can if necessary contain other solvents such as ethanol in addition to the active substance and water. The quantity of ethanol is limited, as a function of the dissolving properties of the active substances, by the fact that the active substance can be precipitated out of the aerosol preparation at excessively high concentrations. Additives for stabilizing the solution such as pharmacologically acceptable preservatives, e.g., ethanol, phenol, cresol, or paraben, pharmacologically acceptable acids, basis or buffers for adjusting the pH or surfactants are also possible. Moreover, in order to stabilize the solution or improve the quality of the aerosol, it is possible to add a metal chelating agent such as EDTA. In order to improve the solubility and/or stability of the active substance in the aerosol preparation, amino acids such as aspartic acid, glutamic acid and particularly prolene may be added.

In addition to interferons, superoxide dismutase and insulin, the preferred active substances in the pharmaceutical preparations according to the invention are as follows: antisense oligonucleotides; orexins; erythropoietin; tumor necrosis factor-alpha; tumor necrosis factor-beta; G-CSF (granulocyte colony stimulating factor); GM-CSF (granulocyte-macrophage colony stimulating factor); annexins; calcitonin; leptins; parathyrin; parathyrin fragment; interleukins, such as interleukin 2, interleukin 10, or interleukin 12; soluble ICAM (intercellular adhesion molecule); somatostatin; somatotropin; tPA (tissue plasminogen activator); TNK-tPA; tumor-associated antigens (as peptide, protein, or DNA); peptide bradykinin antagonists; urodilatin; GHRH (growth hormone releasing hormone); CRF (corticotropin releasing factor); EMAP II; heparin; soluble interleukin receptors such as sIL-1 receptor; vaccines, such as hepatitis vaccine or measles vaccine; antisense polynucleotides; and transcription factors.

What is claimed is:

1. A process for nebulizing an aqueous composition comprising insulin in a concentration of from 25 mg/mL to 100 mg/mL into inhalable droplets, the process comprising:
(a) placing a quantity of the aqueous composition in a chamber having at least one nozzle; and
(b) forcing the composition from the chamber under a pressure of between 100 bar and 500 bar, through at least one of the nozzles to form at least one jet of inhalable droplets of the aqueous composition with an average particle size of less than 10 μm.

2. The process according to claim 1, wherein the nozzle has a hydraulic diameter of 1 μm to 12 μm.

3. The process according to claim 1, wherein the nozzle is a round or non-round opening of 10 microns or less.

4. The process according to claim 1, wherein the concentration of insulin in the aqueous composition is greater than 30 mg/mL.

5. The process according to claim 1, wherein the aqueous composition further comprises one or more adjuvants from the group consisting of surfactants, emulsifiers, stabilizers, permeation enhancers, and preservatives.

6. The process according to claim 1, wherein the aqueous composition further comprises an amino acid.

7. The process according to claim 6, wherein the amino acid is selected from proline, aspartic acid, or glutamic acid.

8. The process according to claim 1, wherein the aqueous composition has a viscosity of up to 1600×10⁻⁶ Pa·s at 25° C.

9. The process according to claim 1, wherein the aqueous composition has a viscosity of between 900×10⁻⁶ and 1100×10⁻⁶ Pa·s at 25° C.

10. The process according to claim 1, wherein the aqueous composition has a viscosity of between 900×10⁻⁶ and 1600×10−6 Pa·s at 25° C.

11. The process according to claim 1, wherein the aqueous composition has a viscosity of between 950×10⁻⁶ and 1300×10⁻⁶ Pa·s at 25° C.

12. The process according to claim 1, wherein the quantity of the aqueous composition placed in the chamber is between 10 μL and 20 μL.

13. The process according to claim 1, wherein the chamber has two nozzles that are so directed that the two jets produced meet and thereby nebulize the aqueous composition.

14. A process for administering insulin by inhalation to a patient in need thereof, the process comprising nebulizing between 10 μL and 50 μL of an aqueous composition comprising between 20 mg/mL and 90 mg/mL of insulin to form inhalable droplets of the aqueous composition in a single application, and wherein the inhalable droplets are then inhaled by the patient.

15. The process according to claim 14, wherein between 10 μL and 20 μL of the aqueous composition comprising between 25 mg/mL and 60 mg/mL of insulin are nebulized.

16. The process of claim 14, wherein the aqueous composition comprises between 30 mg/mL and 90 mg/mL of insulin and the average particle size of the inhalable droplets is less than 10 μm.

17. The process of claim 14, wherein the aqueous composition comprises between 25 mg/mL and 60 mg/mL of insulin and the average particle size of the inhalable droplets is less than 10 μm.

18. The process of claim 14, wherein the aqueous composition comprises between 25 mg/mL and 60 mg/mL of insulin and the average particle size of the inhalable droplets is less than 10 μm, and wherein from 10 μL to 50 μL of solution are administered to the patient.

19. The process of claim 1, wherein the process is performed using a handheld nebulizer.
